(19) United States
(12) Patent Application Publication
(10) Pub. No.: US 2010/0087867 A1
(43) Pub. Date: Apr. 8, 2010

(54) FASTENER ASSEMBLY THAT FASTENS TO PEDICLE SCREW

(76) Inventors: Assaf Klein, Kibbutz Hama'apil (IL); Eyal Zilberberg, Kfar Yona (IL)

Correspondence Address: David Klein;DEKEL PATENT LTD., Beit HaRof'im, 18 Menuha VeNahala Street, Room 27, Rehovot, IL

(21) Appl. No.: 12/361,547

(22) Filed: Jan. 29, 2009

Related U.S. Application Data
(63) Continuation-in-part of application No. 12/244,781, filed on Oct. 3, 2008.

(51) Int. Cl. A61B 17/70; A61B 17/88

(52) U.S. Cl. 606/278; 606/279

(57) ABSTRACT

A fastener assembly including an installed pedicle screw including a fastener head to which a prosthetic member is secured, a fastening portion fastened to the fastener head over the prosthetic member, and a connector element that extends from the fastening portion, the connector element being connected to another spinal structure.

### CROSS REFERENCE TO OTHER APPLICATIONS

This application is a continuation-in-part of U.S. patent application Ser. No. 12/244,781, filed Oct. 3, 2008, and claims priority therefrom.

### FIELD OF THE INVENTION

This invention relates generally to bone fasteners, such as a pedicle screw assembly, and particularly to a fastener assembly that fastens to pedicle screws that have already been installed in spinal structure.

### BACKGROUND OF THE INVENTION

Pedicle screw assemblies are well known in the art and may be used for connecting vertebrae or other spinal structure to rods in spinal surgery. For example, pedicle screw assemblies are known that incorporate a ball joint at the connection to the rod to allow the surgeon some flexibility in placing the screws. Tightening a nut on the screw compresses the ball joint components to lock the angular position of the ball joint.

### SUMMARY OF THE INVENTION

The present invention seeks to provide a fastener assembly that fastens to pedicle screws that have already been installed in spinal structure, as is described more in detail hereinbelow. The fastener assembly of the present invention may be used to fasten different elements to the pedicle screws, such as but not limited to, fusion rods between adjoining vertebrae or crossbars and other connections from the pedicle screws to any suitable portion of the spine in the patient.

The present invention provides the possibility of connecting with cross connectors to fusion systems directly to the pedicle screw and not between the fusion rods as in current prior art systems.

There is thus provided in accordance with an embodiment of the invention a fastener assembly including an installed pedicle screw including a fastener head to which a prosthetic member is secured, a fastening portion fastened to the fastener head over the prosthetic member, and a connector element that extends from the fastening portion, the connector element being connected to another spinal structure.

In accordance with an embodiment of the invention the fastening portion includes a wedge ring and the connector element includes a conical ring into which the wedge ring fits, the connector element including a connecting member that extends from the conical ring, wherein the wedge ring is sized to fit over the fastener head and the conical ring wedges the wedge ring onto the fastener head.

Further in accordance with an embodiment of the invention the wedge ring has a conical periphery with a top surface that has a larger outer diameter than a bottom surface thereof, and the conical ring has a top surface with a larger inner diameter than a bottom surface thereof, the larger outer diameter not being greater than the larger inner diameter. A plurality of grooves may be formed in the conical periphery of the wedge ring. One or more of the grooves may be open at the top surface and closed at the bottom surface and one or more other grooves may be closed at the top surface and open at the bottom surface.

In accordance with another embodiment of the invention the fastening portion includes a closed ring and the connector element includes a connecting member that extends from the closed ring, and wherein the existing pedicle screw includes a first mechanical fastener fastened to the fastener head, wherein the closed ring is placed over the first mechanical fastener such that the first mechanical fastener protrudes through the closed ring, and further including a second mechanical fastener that fastens to the first mechanical fastener and is tightened against the closed ring.

In accordance with yet another embodiment of the invention the fastening portion includes an open ring that fits over the fastener head and a mechanical fastener that fastens end portions of the open ring towards each other so as to clamp the open ring onto the fastener head, and the connector element includes a connecting member that extends from the open ring. The end portions of the open ring can be angled at an acute angle with respect to a top surface of the open ring.

In accordance with another embodiment of the invention the fastening portion includes a closed ring that has a base surface, and the fastener assembly further includes a mechanical fastener that has a cap and a threaded stem extending down from the cap that mates with the fastener head, wherein when the mechanical fastener is threaded onto the fastener head, the cap presses against the closed ring which presses against the prosthetic member, thereby achieving a one-step locking mechanism.

In accordance with another embodiment of the invention the fastener assembly includes a plurality of installed pedicle screws, fastener portions and connector elements, and an assembly of cross bars and connecting rods secured to the pedicle screws with the fastener portions and the connector elements.

There is also provided in accordance with an embodiment of the invention a method including fastening a fastening assembly to a fastener head of an existing pedicle screw that has been previously installed in a spinal structure, a prosthetic member being secured to the fastener head, wherein the fastening assembly includes a fastening portion fastened to the fastener head over the prosthetic member and a connector element that extends from the fastening portion, and connecting the connector element to another spinal structure.

### BRIEF DESCRIPTION OF THE DRAWINGS

The present invention will be understood and appreciated more fully from the following detailed description taken in conjunction with the drawings in which:

FIG. 1 is a simplified pictorial illustration of a fastener assembly, constructed and operative in accordance with an embodiment of the present invention, showing two such fastener assemblies with wedge rings and connector elements secured to pedicle screw heads that have previously been installed in the left and right sides of vertebra;

FIGS. 2A and 2B are partially sectional illustrations of the wedge ring and connector element of FIG. 1;

FIG. 3A is an exploded illustration of the wedge ring, the connector element and a fastening tool used to secure the fastener assembly on an existing pedicle screw, in accordance with an embodiment of the present invention;

FIG. 3B is a pictorial illustration of the wedge ring and connector element mounted in the fastening tool;

FIGS. 3C and 3D are pictorial illustrations of the fastening tool with the wedge ring and connector element placed over the existing pedicle screw;

FIGS. 3E and 3F are pictorial illustrations of using the fastening tool to secure the fastening assembly of FIG. 1 on to the existing pedicle screw;

FIG. 4 is a simplified pictorial illustration of a fastener assembly, constructed and operative in accordance with another embodiment of the present invention, showing two such fastener assemblies with closed rings and connector elements secured to pedicle screw heads that have previously been installed in the left and right sides of vertebra;

FIG. 5 is a partially sectional illustration of the closed ring and connector element of FIG. 4;

FIG. 6A is a pictorial illustration of fastening a set screw of the fastener assembly of FIG. 4 on an existing pedicle screw, in accordance with an embodiment of the present invention;

FIG. 6B is a pictorial illustration of placing the connector element of the fastener assembly of FIG. 4 on the set screw;

FIGS. 6C and 6D are pictorial illustrations of a nut of the fastener assembly of FIG. 4 mounted in a fastening tool used to secure the fastener assembly on an existing pedicle screw, in accordance with an embodiment of the present invention;

FIG. 6E is a pictorial illustration of feeding a torque handle through a counter torque handle of the fastening tool;

FIG. 6F is a pictorial illustration of using the fastening tool to secure the fastening assembly of FIG. 4 on to the existing pedicle screw;

FIG. 7 is a simplified pictorial illustration of a fastener assembly, constructed and operative in accordance with yet another embodiment of the present invention, showing two such fastener assemblies with open rings and connector elements secured to pedicle screw heads that have previously been installed in the left and right sides of vertebra;

FIG. 8 is a more detailed illustration of the open ring and connector element of FIG. 7;

FIG. 9A is a pictorial illustration of fastening a set screw of the existing pedicle screw while the fastener assembly of FIG. 7 is mounted on the existing pedicle screw, in accordance with an embodiment of the present invention;

FIG. 9B is a pictorial illustration of a tool used to fasten the set screw of the existing pedicle screw;

FIG. 9C is a pictorial illustration of aligning the fastener assembly of FIG. 7 on the existing pedicle screw, in accordance with an embodiment of the present invention;

FIG. 9D is a pictorial illustration of a tool used to align the fastener assembly of FIG. 7 on the existing pedicle screw;

FIG. 9E is a pictorial illustration of tightening a set screw of the fastener assembly of FIG. 7 on the existing pedicle screw;

FIGS. 10A and 10B are simplified illustrations of a fastener assembly, constructed and operative in accordance with another embodiment of the present invention, including a closed ring and connector element secured to a previously installed pedicle screw head; and

FIG. 11 is a simplified illustration of an assembly of cross bars and connecting rods secured to existing pedicle screws with any of the above-described embodiments of the invention.

### DETAILED DESCRIPTION OF EMBODIMENTS

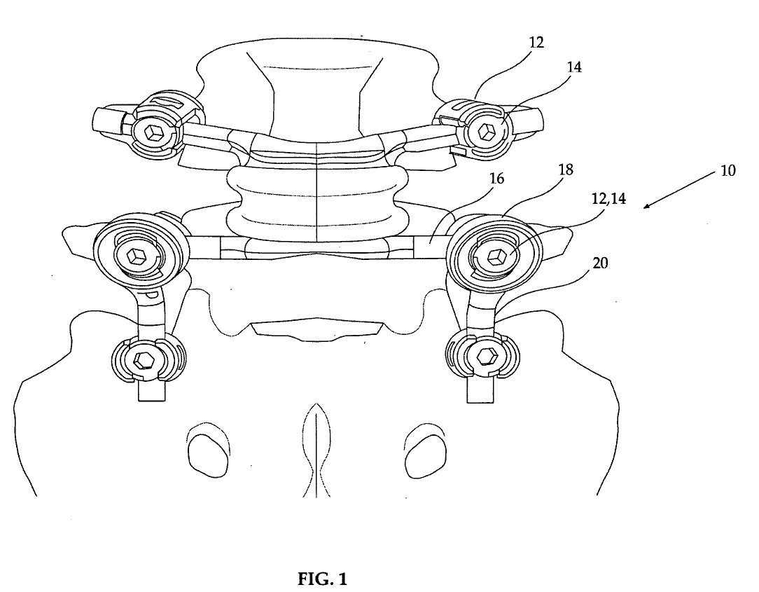

Reference is now made to FIG. 1, which illustrates a fastener assembly 10, constructed and operative in accordance with a non-limiting embodiment of the present invention.

Fastener assembly 10 includes one or more existing pedicle screws 12 (in the illustrated embodiment left and right screws are shown) that have been previously installed in a spinal structure (e.g., pedicles, articular processes, etc.), referred to in the specification and claims as “an installed pedicle screw”. The pedicle screw 12 includes a fastener head 14 to which a prosthetic member 16 is secured. Pedicle screw 12 may be a polyaxial pedicle screw, wherein fastener head 14 is a polyaxial head. In the non-limiting illustrated embodiment, prosthetic member 16 are the prongs of a TOPS (total posterior spine implant) brand spinal prosthesis, commercially available from the present assignee and described in U.S. Pat. No. 7,011,685, the disclosure of which is incorporated herein by reference.

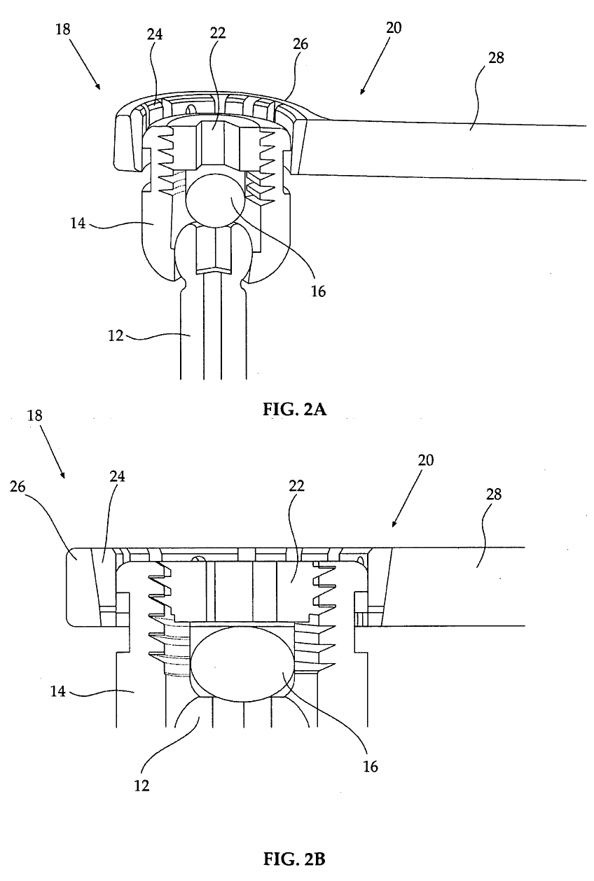

Reference is now made to FIGS. 2A and 2B. A set screw 22 is screwed in the female threads of fastener head 14 and clamps down on prosthetic member 16. A fastening portion 18 (described more in detail below) is fastened to the fastener head 14 over the prosthetic member 16. A connector element 20 extends from fastening portion 18. As seen in FIG. 1, connector element 20 is connected to another spinal structure (e.g., adjacent lumbar vertebra).

In accordance with an embodiment of the invention, fastening portion 18 includes a wedge ring 24 and connector element 20 includes a conical ring 26 into which wedge ring 24 fits. Connector element 20 includes a connecting member 28 that extends from conical ring 26, such as a rod, bar, plate and the like. Wedge ring 24 is sized to fit over fastener head 14 and conical ring 26 wedges wedge ring 24 onto fastener head 14.

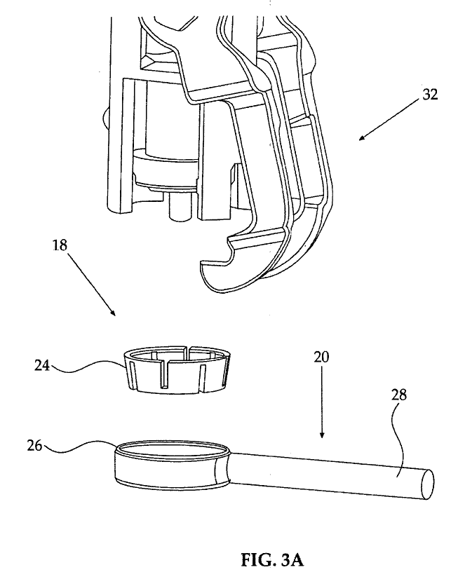

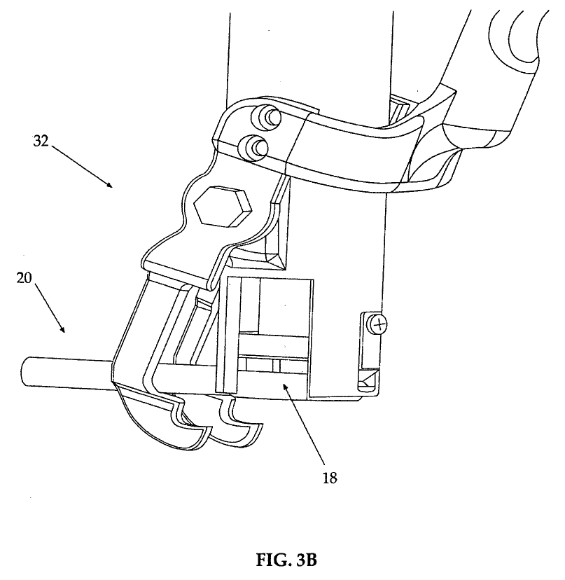

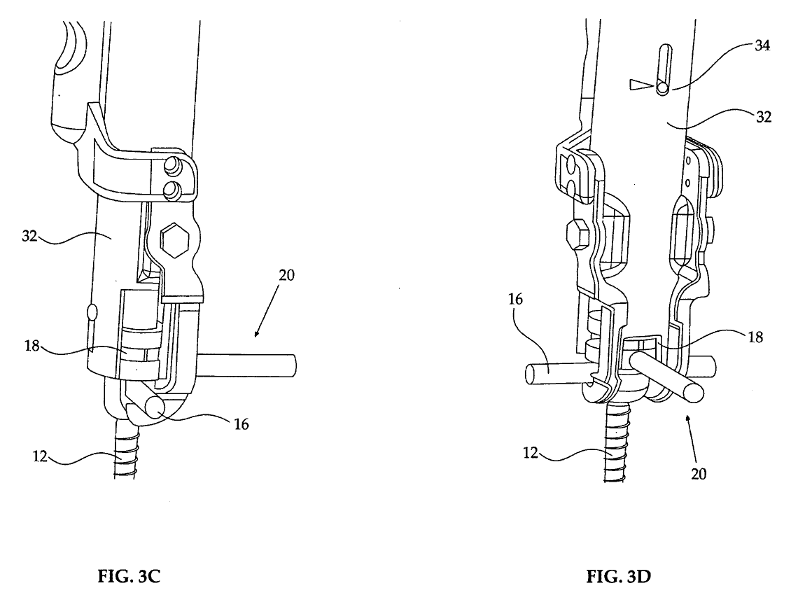

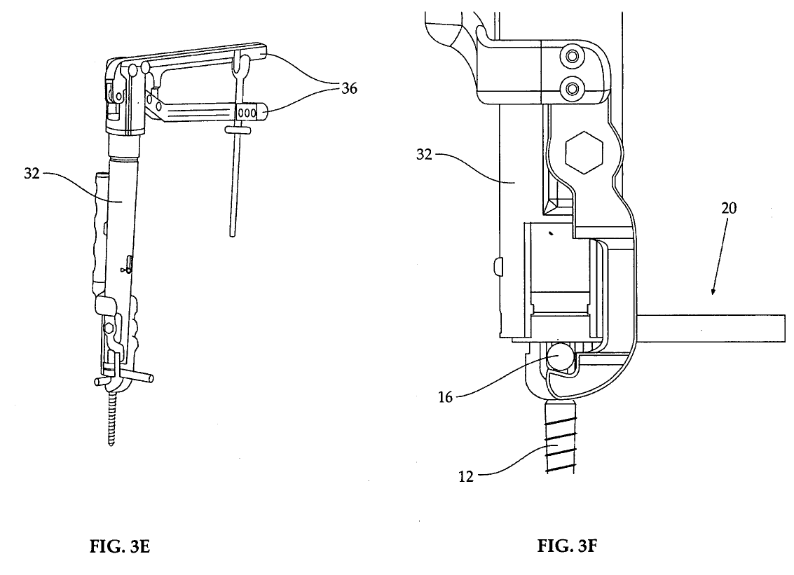

As seen in FIG. 2B, wedge ring 24 has a conical periphery with a top surface that has a larger outer diameter than a bottom surface thereof, and conical ring 26 has a top surface with a larger inner diameter than a bottom surface thereof, the larger outer diameter of wedge ring 24 not being greater than the larger inner diameter of conical ring 26. As seen in FIG. 3A, a plurality of grooves 30 may be formed in the conical periphery of wedge ring 24. One or more of the grooves 30 may be open at the top surface and closed at the bottom surface and one or more other grooves 30 may be closed at the top surface and open at the bottom surface.

The fastener assemblies of the present invention may be constructed of any suitable, rigid, medically safe material, such as but not limited to, stainless steel alloy (e.g., AISI 316L), titanium or titanium alloy or chrome cobalt alloy, PEEK, shape memory alloys or polymers, and any combination thereof.

Reference is now made to FIGS. 3A-3F, which illustrate installing wedge ring 24 and connector element 20 on the pedicle screw 12.

A fastening tool 32 is used to secure the fastener assembly on the existing pedicle screw. FIGS. 3A and 3B show wedge ring 24 and connector element 20 inserted in jaws of fastening tool 32. FIGS. 3C and 3D show fastening tool 32 with wedge ring 24 and connector element 20 placed over the existing pedicle screw 14. The fastening tool 32 has an alignment indicator 34 that indicates the proper alignment of connector element 20 with the existing prosthetic member 16.

FIGS. 3E and 3F show using the fastening tool 32 to secure fastening assembly 10 on to the existing pedicle screw 10. Handles 36 of fastening tool 32 may be squeezed so that conical ring 26 wedges wedge ring 24 onto fastener head 14 as described above with reference to FIGS. 2A-2B.

The set screw of the existing pedicle screw may be left loose or loosened somewhat during installation of the fastener assembly on the existing pedicle screw and afterwards tightened to the recommended torque.

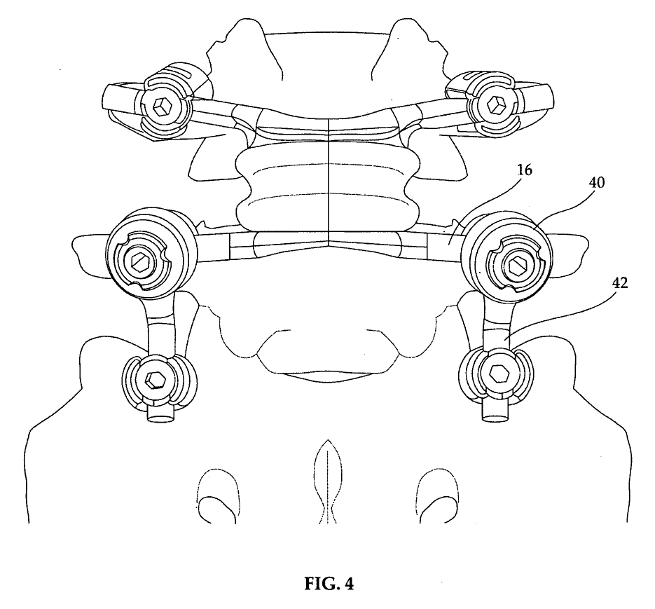

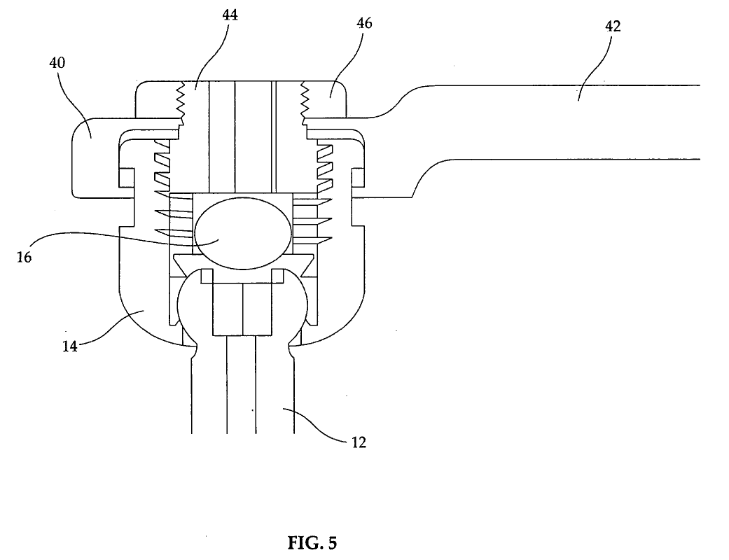

Reference is now made to FIGS. 4 and 5. In accordance with another embodiment of the invention the fastening portion of the fastening assembly includes a closed ring 40 and the connector element includes a connecting member 42 that extends from closed ring 40. The existing pedicle screw 12 includes a first mechanical fastener 44 (such as but not limited to, an elongate set screw with a central through hole) fastened to fastener head 14. Closed ring 40 is placed over the first mechanical fastener 44 such that the first mechanical fastener 44 protrudes through closed ring 40. A second mechanical fastener 46 (such as but not limited to, a nut) fastens to the first mechanical fastener 44 and is tightened against closed ring 40, thereby securing the fastening portion of the fastening assembly to the existing pedicle screw 12.

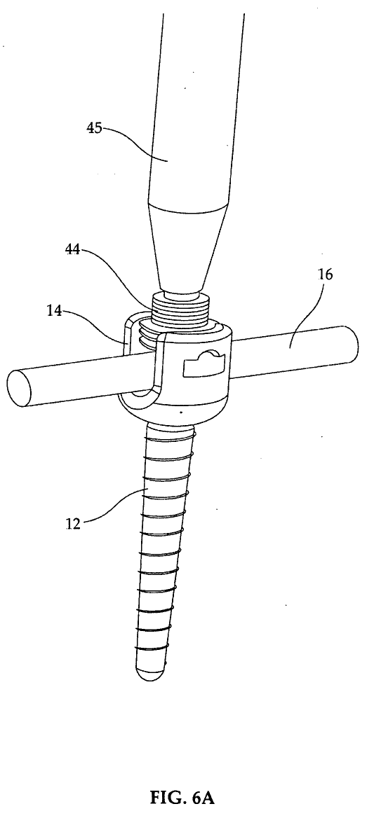

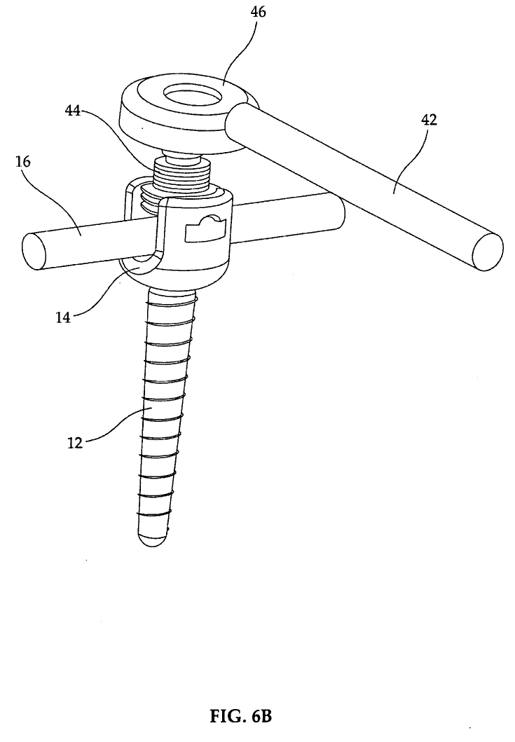

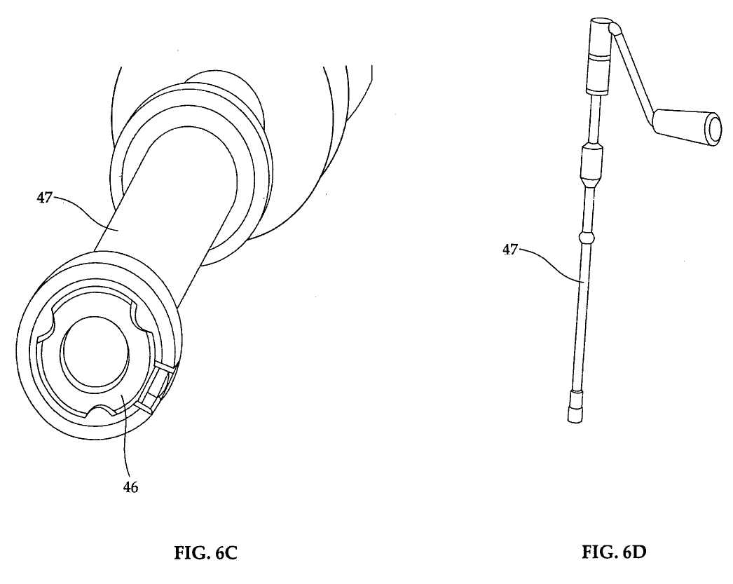

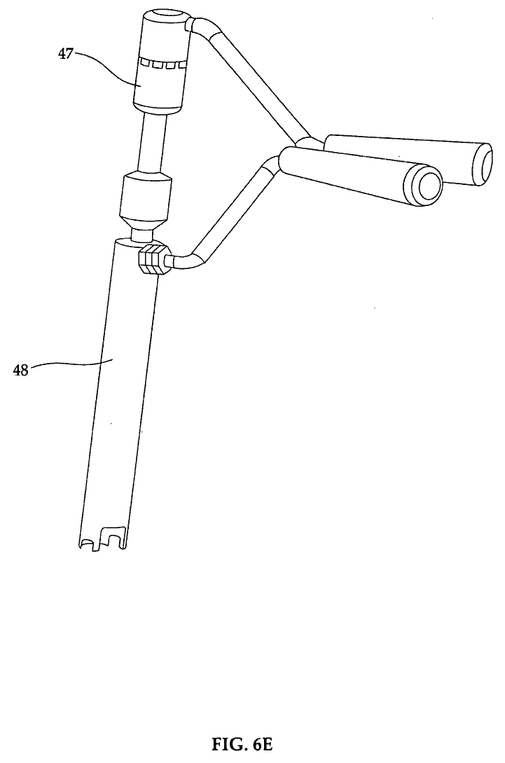

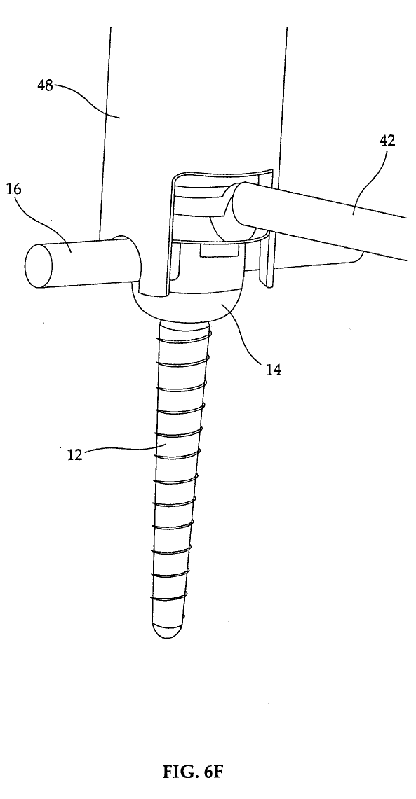

Reference is now made to FIGS. 6A-6F. In FIG. 6A, a tool 45 is used to screw the first mechanical fastener 44 on the fastener head 14 against the prosthetic member 16. In FIG. 6B, the closed ring 40 of the connector element is placed over first mechanical fastener 44 (first mechanical fastener 44 goes through the hole of the closed ring 40). The first mechanical fastener 44 may be further tightened a little at this time.

FIGS. 6C and 6D illustrate second mechanical fastener 46 mounted in a fastening tool (torque handle tool) 47 used to secure the fastener assembly on the existing pedicle screw. FIG. 6E illustrates feeding the torque handle tool 47 through a counter torque handle 48. As seen in FIG. 6F, the counter torque handle 48 is used to hold the fastening assembly in proper alignment on the existing pedicle screw 12. Torque handle tool 47 is used to tighten second mechanical fastener 46 against closed ring 40, thereby securing the fastening portion of the fastening assembly to the existing pedicle screw 12.

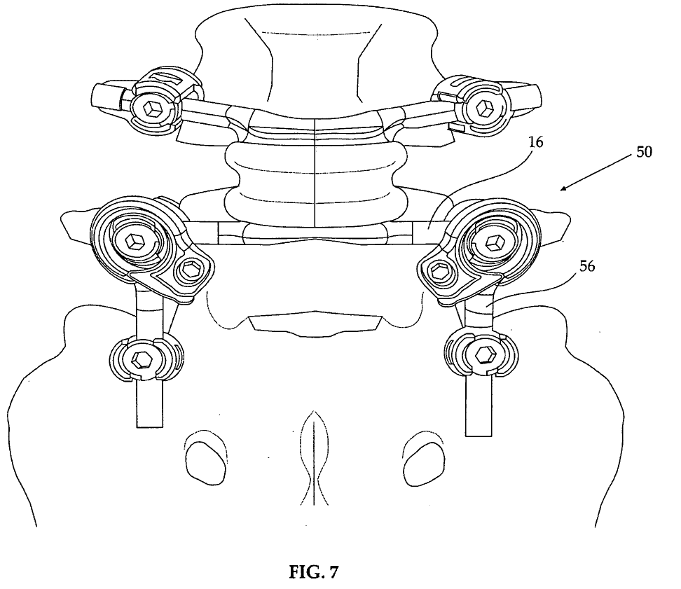

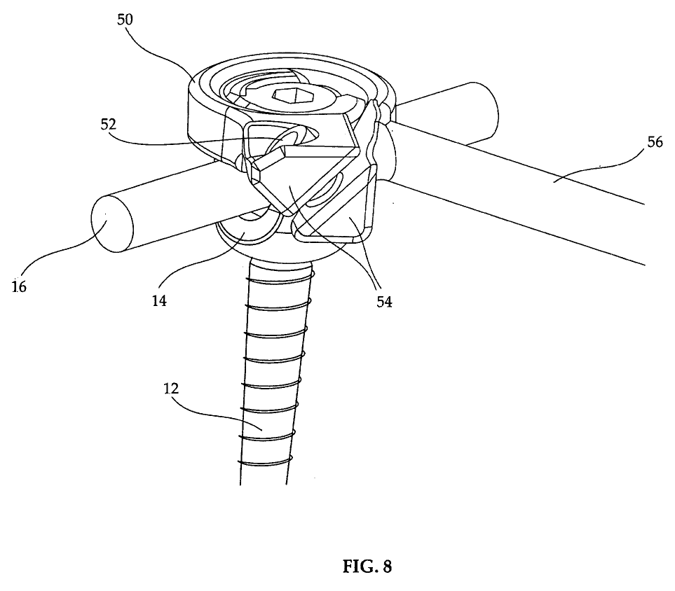

Reference is now made to FIGS. 7 and 8. In accordance with yet another embodiment of the invention the fastening portion includes an open ring 50 that fits over the fastener head 14 and a mechanical fastener 52 that fastens end portions 54 of the open ring 50 towards each other so as to clamp the open ring 50 onto the fastener head 14. The connector element includes a connecting member 56 that extends from open ring 50. The end portions 54 of the open ring 50 can be angled at an acute angle with respect to a top surface of the open ring 50.

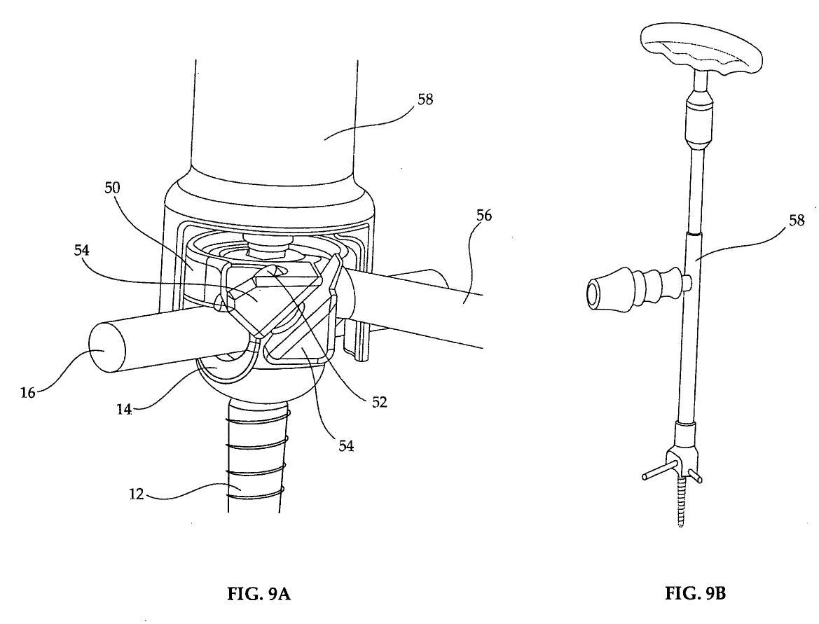

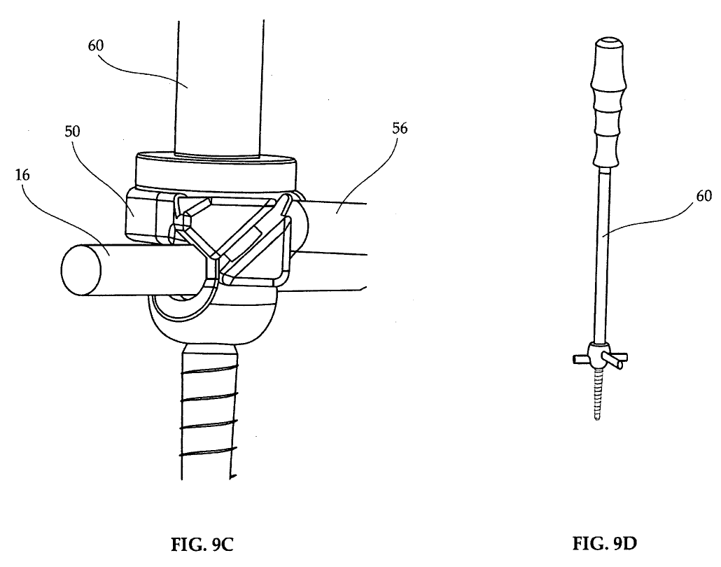

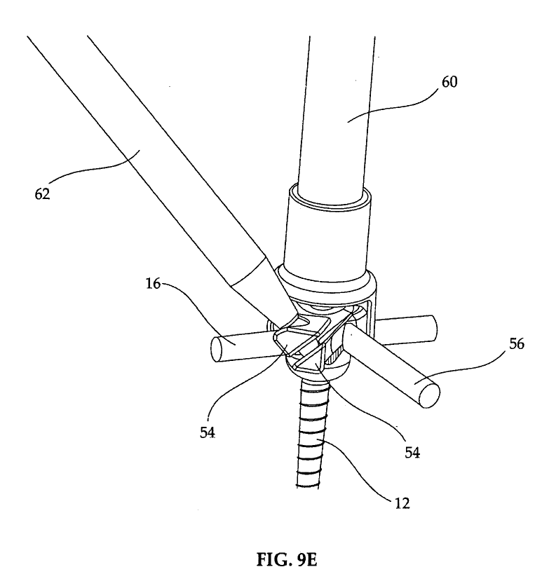

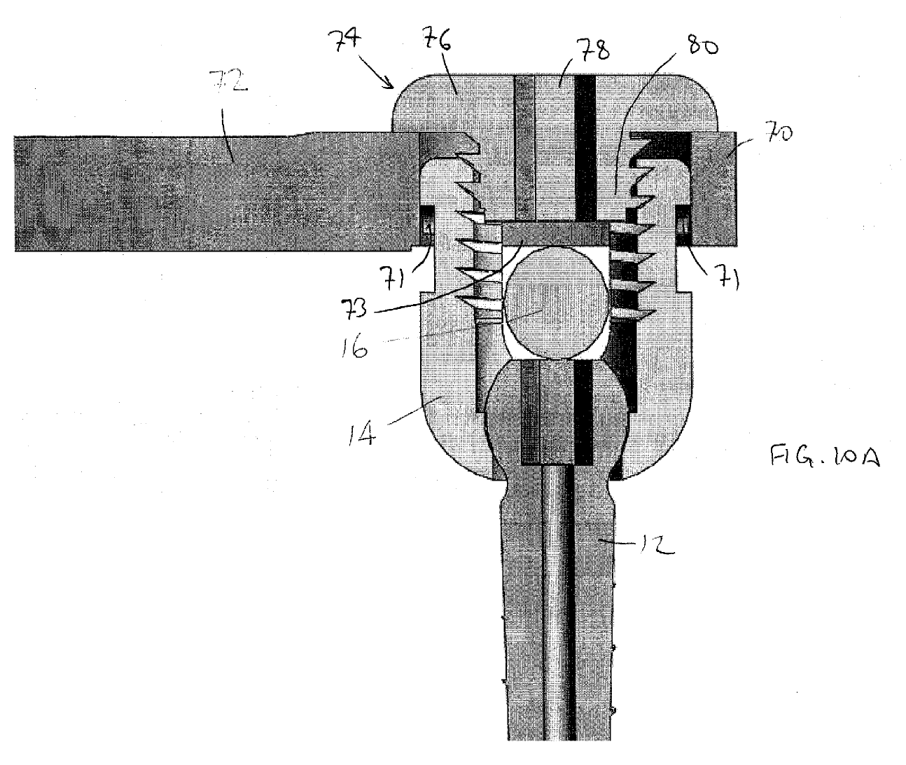

Reference is now made to FIGS. 9A-9E. In FIG. 9A, a fastening tool 58 (also shown in FIG. 9B) is used to fasten the set screw of the existing pedicle screw while the fastener assembly of FIG. 7 is mounted on the existing pedicle screw.

In FIG. 9C, a dedicated alignment tool 60 (also shown in FIG. 9D) is used to align the fastener assembly of FIG. 7 on the existing pedicle screw 12.

In FIG. 9E, a tightening tool 62 is used to tighten the mechanical fastener of the fastener assembly of FIG. 7 so as to clamp the open ring 50 onto the fastener head 14.

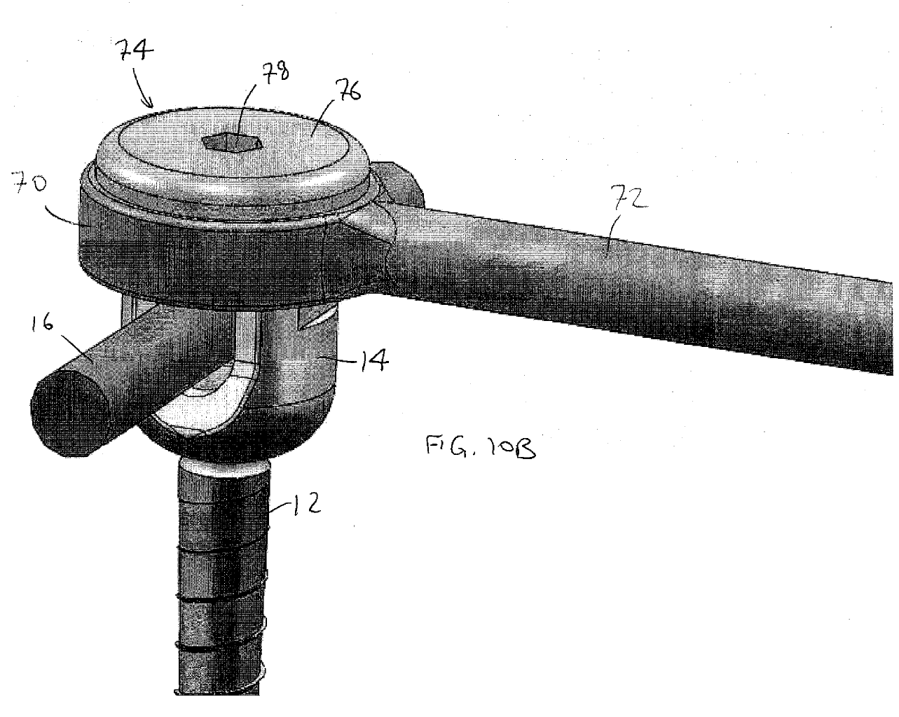

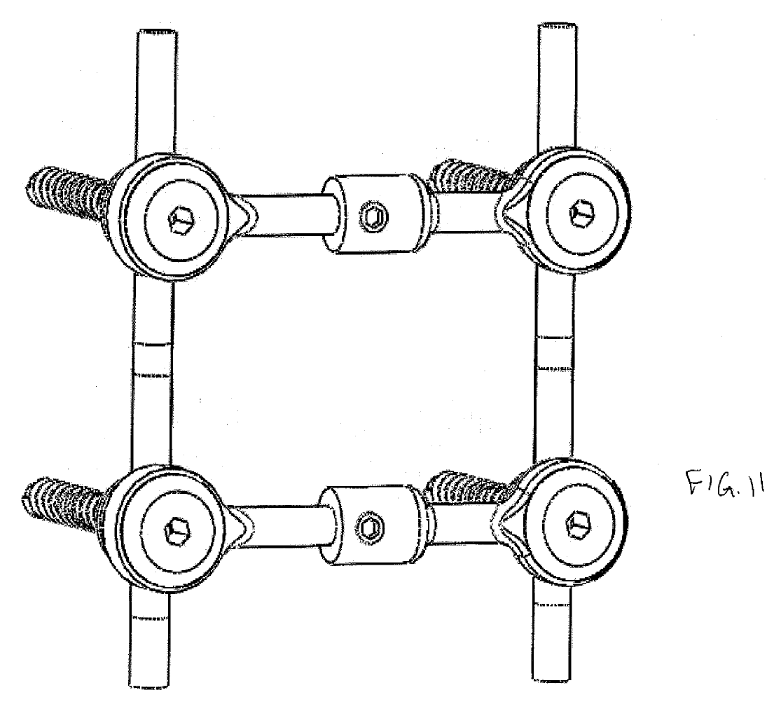

Reference is now made to FIGS. 10A and 10B. In accordance with another embodiment of the invention the fastening portion of the fastening assembly includes a closed ring 70 and the connector element includes a connecting member 72 that extends from closed ring 70. Closed ring 70 has two apertures 71 for receiving therethrough the two upright portions of fastener head 14. Closed ring 70 also has a lower base surface 73 situated between the two apertures 71. A mechanical fastener 74 is provided that has a cap 76 with a tool interface 78, such as but not limited to, a hexagonal hole for accepting an Allen wrench and the like, and a threaded stem 80 extending down from cap 76. Threaded stem 80 mates with the female (internal) threads of fastener head 14. Cap 76 is sized such that when mechanical fastener 74 is threaded onto fastener head 14, cap 76 presses against closed ring 70, which presses against the prosthetic member 16 at the same time and achieve a one-step locking mechanism. (Alternatively, at the same time, threaded stem 80 presses base surface 73 of closed ring 70 against prosthetic member 16, thereby securing prosthetic member 16 against the top of pedicle screw 12.)

FIG. 11 illustrates an assembly of cross bars and connecting rods secured to existing pedicle screws with any of the above-described embodiments of the invention.

It is appreciated that various features of the invention which are, for clarity, described in the contexts of separate embodiments, may also be provided in combination in a single embodiment. Conversely, various features of the invention which are, for brevity, described in the context of a single embodiment, may also be provided separately or in any suitable subcombination.

What is claimed is:

1. A fastener assembly comprising:
an installed pedicle screw comprising a fastener head to which a prosthetic member is secured;
a fastening portion fastened to said fastener head over said prosthetic member; and
a connector element that extends from said fastening portion, said connector element being connected to another spinal structure.

2. The fastener assembly according to claim 1, wherein said fastening portion comprises a wedge ring and said connector element comprises a conical ring into which said wedge ring fits, said connector element comprising a connecting member that extends from said conical ring, wherein said wedge ring is sized to fit over said fastener head and said conical ring wedges said wedge ring onto said fastener head.

3. The fastener assembly according to claim 2, wherein said wedge ring has a conical periphery with a top surface that has a larger outer diameter than a bottom surface thereof, and said conical ring has a top surface with a larger inner diameter than a bottom surface thereof, said larger outer diameter not being greater than said larger inner diameter.

4. The fastener assembly according to claim 3, wherein a plurality of grooves are formed in said conical periphery of said wedge ring.

5. The fastener assembly according to claim 4, wherein at least one of said grooves is open at the top surface and closed at the bottom surface and at least another groove is closed at the top surface and open at the bottom surface.

6. The fastener assembly according to claim 1, wherein said fastening portion comprises a closed ring and said connector element comprises a connecting member that extends from said closed ring, and wherein said existing pedicle screw comprises a first mechanical fastener fastened to the fastener head, wherein said closed ring is placed over said first mechanical fastener such that said first mechanical fastener protrudes through said closed ring, and further comprising a second mechanical fastener that fastens to said first mechanical fastener and is tightened against said closed ring.

7. The fastener assembly according to claim 6, wherein said closed ring is not flush with said connecting member.

8. The fastener assembly according to claim 1, wherein said fastening portion comprises an open ring that fits over said fastener head and a mechanical fastener that fastens end portions of said open ring towards each other so as to clamp said open ring onto said fastener head, and said connector element comprises a connecting member that extends from said open ring.

9. The fastener assembly according to claim 8, wherein said end portions of said open ring are angled at an acute angle with respect to a top surface of said open ring.

10. The fastener assembly according to claim 1, wherein said fastening portion comprises a closed ring that has a base surface, and the fastener assembly further comprises a mechanical fastener that has a cap and a threaded stem extending down from said cap that mates with said fastener head, wherein when said mechanical fastener is threaded onto said fastener head, said cap presses against said closed ring which presses against said prosthetic member.

11. The fastener assembly according to claim 1, further comprising a plurality of installed pedicle screws, fastener portions and connector elements, and an assembly of cross bars and connecting rods secured to said pedicle screws with said fastener portions and said connector elements.

12. A method comprising:
fastening a fastening assembly to a fastener head of an installed pedicle screw, a prosthetic member being secured to said fastener head, wherein said fastening assembly comprises a fastening portion fastened to said fastener head over said prosthetic member and a connector element that extends from said fastening portion; and
connecting said connector element to another spinal structure.
